(19) United States
(12) Patent Application Publication
(10) Pub. No.: US 2026/0364330 A1
(43) Pub. Date: Nov. 19, 2026

(54) INTRAVENOUS TUBING UPPER FITMENT

(71) Applicant: CareFusion 303, Inc., San Diego, CA (US)

(72) Inventors: Ranjith Sankar, Chennai (IN); Manikandan Saravanan, Tiruchengode Taluk (IN)

(21) Appl. No.: 19/210,294

(22) Filed: May 16, 2025

(51) Int. Cl. A61M 39/10; A61M 5/142

(52) U.S. Cl. CPC A61M 39/10; A61M 5/142

(57) ABSTRACT

An intravenous tubing fitment is provided including a body having a first end, a bottom flange adjacent to the first end, a top flange adjacent to a second end, and a middle portion extending between the bottom flange and the top flange. The first end of the body is configured to couple to a first tubing and the second end of the body is configured to receive a second tubing. The body is configured to be received into a mating recess of a fluid delivery device such that the bottom flange is received into a slot of the mating recess and the body can then be moved into an inner bore of the mating recess to resist unintended movement of the intravenous tubing fitment relative to the fluid delivery device.

### TECHNICAL FIELD

The present disclosure generally relates to an intravenous (IV) set or infusion pump flow control, and in particular an IV tubing fitment for securing an IV tube to a fluid delivery device.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. IV sets are loaded into a medical device, such as an infusion pump, which controls the flow rate through the tubing. Due to the designs of existing IV sets, the IV sets can be unintentionally misloaded causing the tubing of the IV sets to be pulled, twisted, entangled, or otherwise interfered with. In some instances, an IV tubing fitment that is coupled to the IV tubing is misloaded into a medical device. IV sets that are misloaded result in portions of the tubing to be reshaped, which in turn causes a change or occludes a flow rate produced by the medical device. A misloaded IV set, or portion thereof, can result in misalignment of the IV set with a pressure sensor of the medical device, and in some instance, damage to the medical device and/or the IV set. For example, IV set that are misloaded can be pulled causing the tubing to stretch and narrow a diameter of the tubing. Misloaded IV sets can result in inaccurate flow rates, uncontrolled flow rates, over infusion, under infusion, and/or a number of other issues.

Thus, it is desirable to provide an IV set that reduces the opportunities for misloading into a medical device.

### SUMMARY

Various implementations of systems, methods, and devices within the scope of the appended claims each have several aspects, no single one of which is solely responsible for the attributes described herein. Without limiting the scope of the appended claims, after considering this disclosure, and particularly after considering the section entitled “Detailed Description” one will understand how the aspects of some implementations are used to prevent an IV set from being misloaded to provide accurate and consistent flow rates. One or more implementations provide an IV tubing fitment including body comprising a first end configured to couple to a first portion of a tubing, a second end configured to receive a second portion of the tubing, a bottom flange adjacent to the first end, and a top flange adjacent to the second end. The body can be received into a mating recess of a fluid delivery device so that the bottom flange is positioned within a slot in the mating recess and engagement of the body against the fluid delivery device resists tilting of the body relative to the fluid delivery device. Thus, the IV tubing fitment can resist misloading or misalignment of the IV tubing fitment with the fluid delivery device.

In one aspect, an intravenous tubing fitment system is provided. The IV tubing fitment system includes an IV tubing fitment and a fluid delivery device. The IV tubing fitment comprises a body having a first end, configured to couple to a first portion of a tubing, a second end, configured to receive a second portion of the tubing, a bottom flange, and a top flange, wherein the bottom flange is adjacent to the first end, and the top flange adjacent to the second end, and the fluid delivery device comprises a housing having an inner surface forming a mating recess comprising a lower ledge, extending radially inward into the mating recess, an upper ledge, extending radially inward into the mating recess, and a slot formed between the lower ledge and the upper ledge. The body of the IV tubing fitment is configured to be received into the mating recess such that the bottom flange is positioned within the slot and a middle portion of the body, between the bottom flange and the top flange extends through a bore of the upper ledge, and wherein engagement of the body against the fluid delivery device resists tilting of the body relative to the fluid delivery device. In some implementations, a method of providing an intravenous tubing fitment system comprises providing an IV tubing fitment comprising a body having a first end, configured to couple to a first portion of a tubing, a second end, configured to receive a second portion of the tubing, a bottom flange, and a top flange, wherein the bottom flange is adjacent to the first end, and the top flange adjacent to the second end, providing a fluid delivery device comprising a housing having an inner surface forming a mating recess comprising a lower ledge, extending radially inward into the mating recess, an upper ledge, extending radially inward into the mating recess, and a slot formed between the lower ledge and the upper ledge, and positioning the body into the mating recess such that the bottom flange is within the slot and a middle portion of the body, between the bottom flange and the top flange extends through a bore of the upper ledge, and engagement of the body against the fluid delivery device resists tilting of the body relative to the fluid delivery device. The foregoing and other features, aspects and advantages of the disclosed implementations will become more apparent from the following detailed description and accompanying drawings.

### BRIEF DESCRIPTION OF THE DRAWINGS

The accompanying drawings, which are included to provide further understanding of the disclosure and are incorporated in and constitute a part of this specification, illustrate implementations of the disclosure and together with the description serve to explain the principles of the disclosure.

FIG. 1 shows a patient care system, in accordance with various aspects of the present disclosure.

FIG. 2 shows an infusion pump and an IV set, in accordance with various aspects of the present disclosure.

FIG. 3 illustrates an IV tubing fitment, in accordance with some implementations.

FIGS. 4A-4C illustrate different views of the IV tubing fitment in accordance with some implementations.

FIG. 5 illustrates a detail view of an infusion pump, in accordance with some implementations.

FIGS. 6 illustrates a top section view of the fluid delivery device of FIG. 5, in accordance with some implementations.

FIG. 7 illustrates a perspective view loading of an IV tubing fitment into a fluid delivery device, in accordance with some implementations.

FIG. 8 illustrates a front view of an IV tubing fitment loaded into a fluid delivery device, in accordance with some implementations.

FIG. 9 illustrates a top section view of the fluid delivery device and the IV tubing fitment of FIG. 8, in accordance with some implementations.

Like reference numerals refer to corresponding parts throughout the drawings.

### DETAILED DESCRIPTION

The disclosed IV tubing fitment secures an IV set to fluid delivery device such that damage to the IV tubing fitment and the fluid delivery device is resisted or prevented. The IV tubing fitment also resists damage or alteration to tubing of the IV set caused by misloading of the IV tubing fitment into the fluid delivery device.

The IV tubing fitment includes an intuitive design that allows users to readily recognize an orientation in which the IV tubing fitment should be placed within a fluid delivery device to ensure that the IV set is properly loaded. Further, the IV tubing fitment is designed to secure the position of the IV tubing fitment in the fluid delivery device, thereby ensuring proper alignment of the IV set tubing with a pressure sensor(s) and proper alignment of the IV tubing fitment in the fluid delivery device. The IV tubing fitment reduces the chances of the IV set being damaged by being forced into a fluid delivery device and prevents damage to the IV tubing fitment when a door of the fluid delivery device is closed, thereby improving the accuracy and consistency of the flow rate generated by the fluid delivery device.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to an IV tubing fitment configured to be loaded into a fluid delivery device, it is to be understood that this description is only an example of usage and does not limit the scope of the claims. Various aspects of the disclosed IV tubing fitment may be used in any application where it is desirable to secure an IV set or other type of tubing to a fluid delivery device.

The disclosed IV tubing fitment overcomes several challenges discovered with respect to certain conventional IV sets. One challenge with certain conventional IV sets is the misloading of IV sets that can cause damage to the IV tubing fitment and/or a tube of the IV sets. Misloading of an IV set can result in inaccurate, inconsistent, and uncontrollable flow rates, and damage to the IV tubing fitment and/or the fluid delivery device. Because of the inaccuracies or the inconsistencies of caused by misloaded IV sets, thereby potentially harming the patient or decreasing the efficacy of a treatment, the use of conventional IV sets afford limited protection from adverse conditions due to misloading.

Therefore, in accordance with the present disclosure, it is advantageous to provide an IV tubing fitment as described herein that eliminates or substantially reduces the possibility of misloading IV sets. The disclosed IV tubing fitment securely holds an IV set in place and also prevents a fluid delivery device from receiving an IV set if misloaded thereby improving the accuracy and consistency of a generated flow rate. An example of an IV tubing fitment is now described.

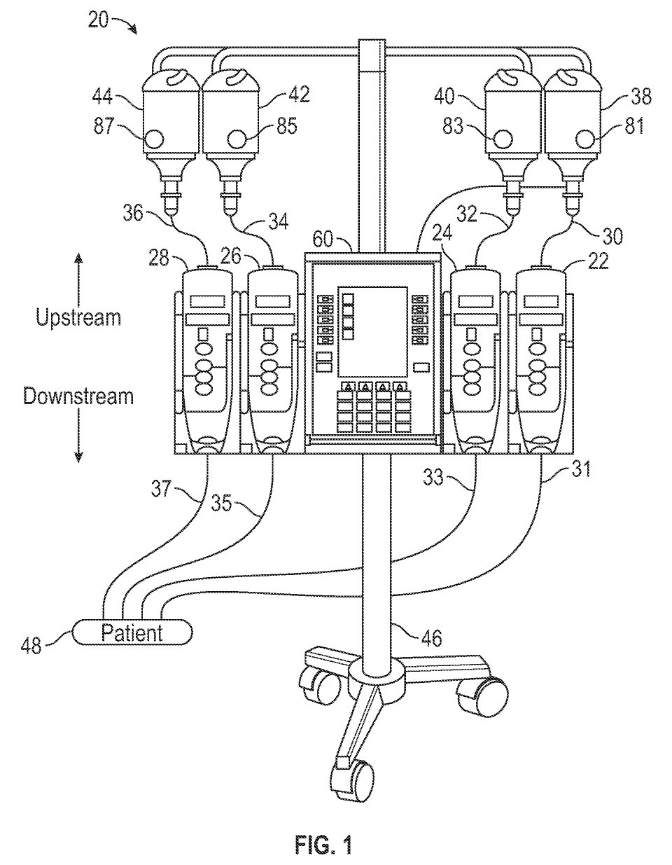

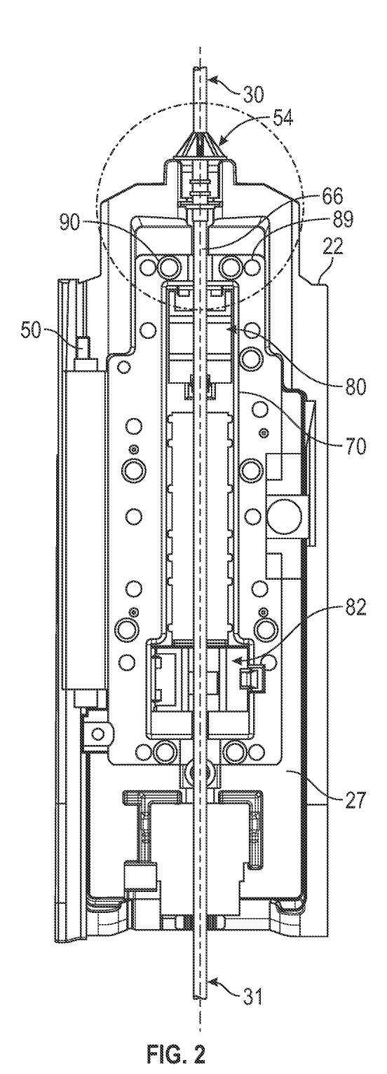

FIG. 1 shows a patient care system, in accordance with various aspects of the present disclosure. The patient care system 20 includes four infusion pumps 22, 24, 26, and 28 each of which is fluidly connected with an upstream fluid line 30, 32, 34, and 36, respectively. Each fluid line 30, 32, 34, and 36 is held in place at a respective infusion pumps 22, 24, 26, and 28 via a respective IV tubing fitment (e.g., IV tubing fitment 54; FIG. 2). Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as medicament bags, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, tabletop, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a controller 60. In some implementations, the controller 60 is communicatively coupled to memory storing one or more instructions for operating the infusion pumps 22, 24, 26, and 28 and/or other collected data as described below. Alternatively, in some implementations, each infusion pump 22, 24, 26, and 28 includes its own controller.

Fluid supplies 38, 40, 42, and 44 can each be coupled to an electronic data tag 81, 83, 85, and 87, respectively, or to an electronic transmitter. Any device or component associated with the infusion system may be equipped with an electronic data tag, reader, or transmitter.

Typically, medical fluid administration sets have more parts than are shown in FIG. 1. Many have check valves, drip chambers, valves with injection ports, connectors, and other devices well known to those skilled in the art. These other devices have not been included in the drawings so as to preserve clarity of illustration.

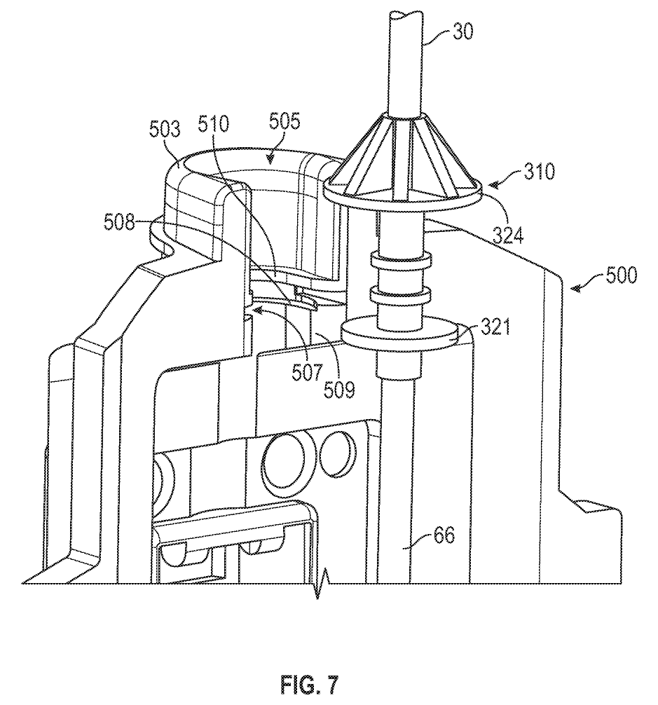

Turning now to FIG. 2, an infusion pump 22 having a housing 27 is shown in perspective view, in accordance with various aspects of the present disclosure. The infusion pump 22 includes a front door (not included for clarity of illustration) that is couplable to a hinge 50. With the front door open, the upstream fluid line 30 and the downstream fluid line 31 are visible in operative engagement with the infusion pump 22. More specifically, the fluid line 30 is coupled to (or received by) an IV tubing fitment 54 that is held in place within a pocket and/or mating recess of the infusion pump 22. The IV tubing fitment 54 is a unitary body including a first end configured to couple to a first portion of a tubing (e.g., tube 66) and a second end opposite the first end coupled to (or configured to receive) a second portion of the tubing (e.g., fluid line 30). The IV tubing fitment 54 includes a bottom flange that, when received by the infusion pump 22, is uniformly mated with a slot of the mating recess of the infusion pump 22. Additional information on the placement of the IV tubing fitment 54 is provided below in reference to FIGS. 7-9.

The IV tubing fitment 54 reduces the chances of an IV set, including the upstream fluid line 30, the tube 66, and the downstream fluid line 31, from being misloaded by preventing the IV tubing fitment from being positioned at an angle or tilted, relative to the infusion pump. The IV tubing fitment 54 also reduces the chances of the door of the infusion pump 22 from closing and damaging the infusion pump housing, the door, and the IV tubing fitment if the IV tubing fitment 54 is not properly mated to the mating recess of the infusion pump 22. For example, a bottom flange of the IV tubing fitment 54 is such that the IV tubing fitment 54 is seated in the slot of the mating recess and lateral, axial, and tilting movement of the IV tubing fitment 54, relative to the infusion pump housing, is resisted. More specifically, in some implementations, the bottom flange of the IV tubing fitment 54 has a diameter that is substantially equal to or less than a diameter of the slot of the mating recess of the infusion pump 22. In some implementations, the IV tubing fitment 54 also includes a top flange that engages against another portion of the housing when the bottom flange is positioned within the mating recess. The features of the IV tubing fitment 54 permits placement of the IV set in the infusion pump when the door is open, thereby providing ease of visualization and convenient manipulation of the IV set by the user, thereby increasing efficiency and the ability to ensure proper placement of the IV set before the door is closed. Thus, by ensuring proper placement of the IV set, the IV tubing fitment 54 prevents a user from closing the door and causing damage to the IV set or the infusion pumps.

Additionally, the IV tubing fitment 54 allows the IV set to rotate about its longitudinal axis (e.g., center line of the upstream fluid line 30) while preventing the IV set from twisting, kinking, and/or entangling. In some implementations, the IV tubing fitment 54 rotates about a longitudinal axis at least 270 degrees in a clockwise and counterclockwise direction. In some implementations, the IV tubing fitment 54 rotates about the longitudinal axis a full 360 degrees in a clockwise and counterclockwise direction. By preventing the IV set from twisting, kinking, and/or entangling, the IV tubing fitment 54 improves the accuracy and consistency of the flow rate be eliminating or reducing any accidental or unintentional occlusion caused by movement of the IV set, as well as potential damage to the IV set. Further, the IV tubing fitment 54 is further configured to center the tubing 66 when the bottom flange of the IV tubing fitment 54 is mated to the mating recess of the infusion pump 22.

The infusion pump 22 directly acts on the tube 66 that connects the upstream fluid line 30 to the downstream fluid line 31 to form a continuous fluid conduit, extending from the respective fluid supply 38 (FIG. 1) to the patient 48, through which fluid is acted upon by the pump to move fluid downstream to the patient. Specifically, a pumping mechanism acts as the flow control device of the pump to move fluid though the conduit. The upstream and downstream fluid lines and/or tube 66 may be coupled to a pump cassette or cartridge that is configured to be coupled to the pump 22.

The type of pumping mechanism may vary and may be for example, a multiple finger pumping mechanism. For example, the pumping mechanism may be of the “four finger” type and includes an upstream occluding finger, a primary pumping finger, a downstream occluding finger, and a secondary pumping finger. The “four finger” pumping mechanism and mechanisms used in other linear peristaltic pumps operate by sequentially pressing on a segment of the fluid conduit by means of the cam-following pumping fingers and valve fingers. The pressure is applied in sequential locations of the conduit, beginning at the upstream end of the pumping mechanism and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

FIG. 2 further shows a downstream pressure sensor 82 included in the pump 22 at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient 48 (FIG. 1) and the flow control device, so that the connection of the correct fluid supply with the correct pump may be verified before any fluid is pumped to the patient.

With reference still to FIG. 2, an upstream pressure sensor 80 may also be included in the pump 22. The upstream pressure sensor is assigned to the flow control device or pumping mechanism and, in this implementation, is further provided as an integral part of the pump 22. It is mounted to the flow control device 70 and is located adjacent and upstream in relation to the flow control device. The upstream pressure sensor is located upstream from the flow control device, that is, at a location between the fluid supply 38 (FIG. 1) and the flow control device, so that the connection of the correct fluid supply with the correct pump may be verified before any fluid is pumped to the patient.

The pump 22 or a portion of the pump 22 may also be equipped with an electronic data tag or data transmitter. For example, as shown in FIG. 2, pump 22 may be equipped with a data tag 89 or a reader device 90 for providing or receiving infusion data. The data reader devices may include RFID readers (or receivers) or other wireless devices that are compatible with the data tags associated with the fluid containers. A data transmitter may transmit interrogation signals to the electronic data tags 81, 83, 85, 87 associated with the fluid containers for obtaining infusion data from those tags. Although referred to as data transmitting devices or RFID tags or RFID transponders, data transmitting devices may also receive or read data and may also be writable.

Typically, medical tubing is a disposable product that is used once and then discarded. The medical tubing may be formed from any suitable material, e.g., soft PVC, silicone, thermoplastic vulcanizate (TPV) (ethylene propylene diene monomer (EPDM) + polypropylene (PP)), thermoplastic polyurethane (TPU), thermoplastic styrenic elastomer (TPS) (styrene-butadiene-styrene (SBS) /styrene-ethylene-butylene-styrene (SEBS) /styrene-isoprene-rubber (SIS) /styrene-ethylene-propylene-styrene (SEPS)) and its blending with polyolefin , thermoplastic polyester elastomer (TPEE) (polyether ester) rubber). As shown in FIG. 2, medical tubing 66 may be inserted into or otherwise engaged by pump 22. Infusion pump 22 may include any of large volume (e.g., at least 100 milliliter of fluid from a single container), patient-controlled analgesia (PCA), ambulatory pump or insulin pump that drive tubing segment(s) to deliver medication or nutrients into a patient’s body in controlled amounts. The medical tubing 66 is compressed when the pump door 50 is closed. With the pump door 50 closed, the medical tubing 66 is constrained within the infusion pump 22 mating recess (by using the IV tubing fitment 54) and directly contacted by the upstream force sensor 80. As discussed above, there are many sources of variation in measuring the force on the medical tubing 66 by the sensor 80.

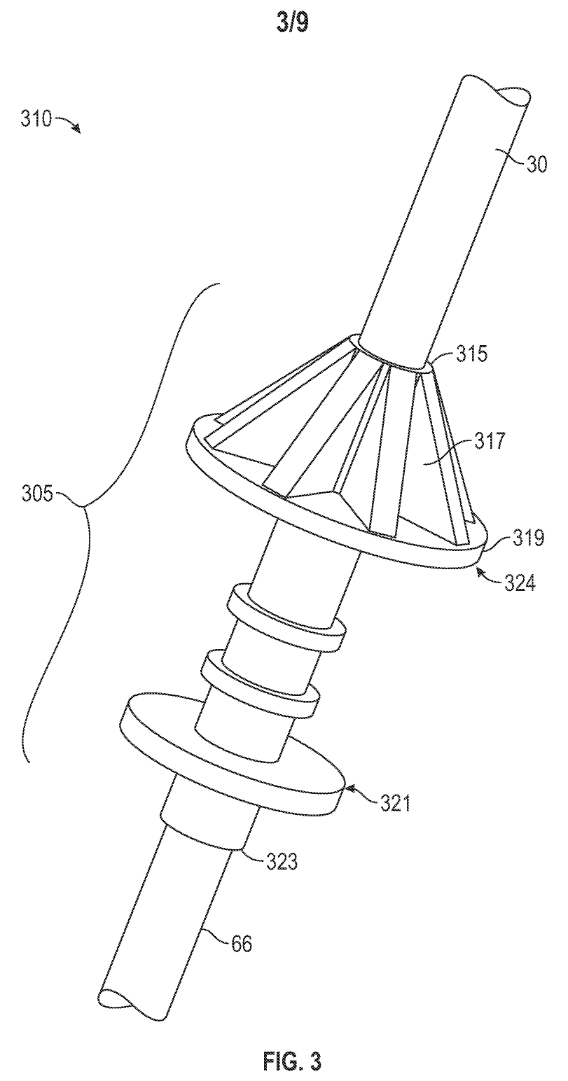

FIG. 3 illustrates an IV tubing fitment, in accordance with some implementations. The IV tubing fitment 310 is an example of the IV tubing fitment 54 described above in reference to FIG. 2. The IV tubing fitment 310 includes a body 305. In some implementations, the body 305 is unitary and can be cast, molded, forged, or 3D printed. The body 305 includes a first end 323 configured to couple to a first portion of a tubing (e.g., tube 66; FIG. 2), a bottom flange 321 is adjacent to the first end 323, and a second end 315 is adjacent to the bottom flange 321 and configured to receive a second portion of the tubing (e.g., upstream fluid line 30; FIG. 2). The bottom flange 321 is positioned between the first end 323 and the second end 315. A top flange 324 is adjacent to the second end 315. The top flange 324 is positioned between the bottom flange 321 and the second end 315.

The body 305 further includes a plurality of wings 317 that extends from the top flange 324 towards an outer surface of the body 305 that is between the top flange 324 and the second end 315. The plurality of wings 317 provide a user an intuitive means of determining the portion of the IV tubing fitment 310 that should be placed within a fluid delivery device (e.g., infusion pump 22; FIG. 2). In some implementations, the plurality of wings 317 do not reach the outer rim 319, and in some implementations, the plurality of wings 317 extend to an outer rim 319 along the perimeter of the top flange 324.

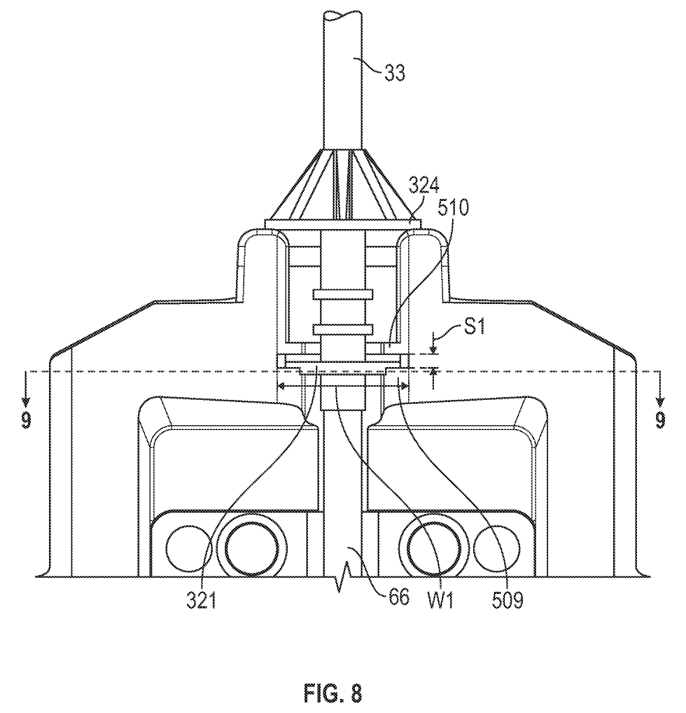

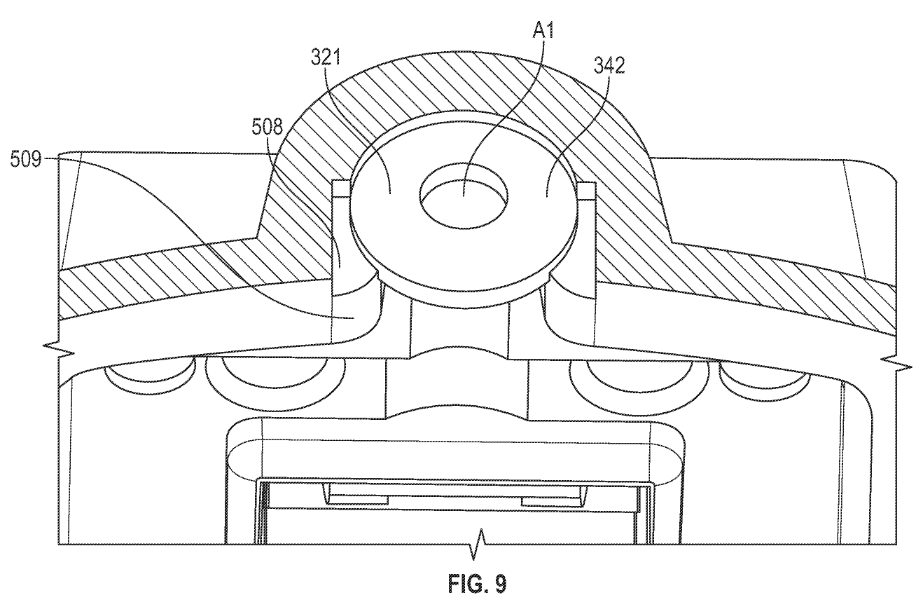

The bottom flange 321 allowing the IV tubing fitment 310 to couple with the fluid delivery device restricting the IV tubing fitment 310 movement in the axial and lateral directions while also allowing the IV tubing fitment 310 to rotate about the longitudinal axis. More specifically, the bottom flange 321 is configured to engage with the housing of the fluid delivery device such that when the IV tubing fitment 310 is engaged with the mating recess of the fluid delivery device, movement of the body 305 is restricted in at least axial direction as described below in reference to FIGS. 8 and 9.

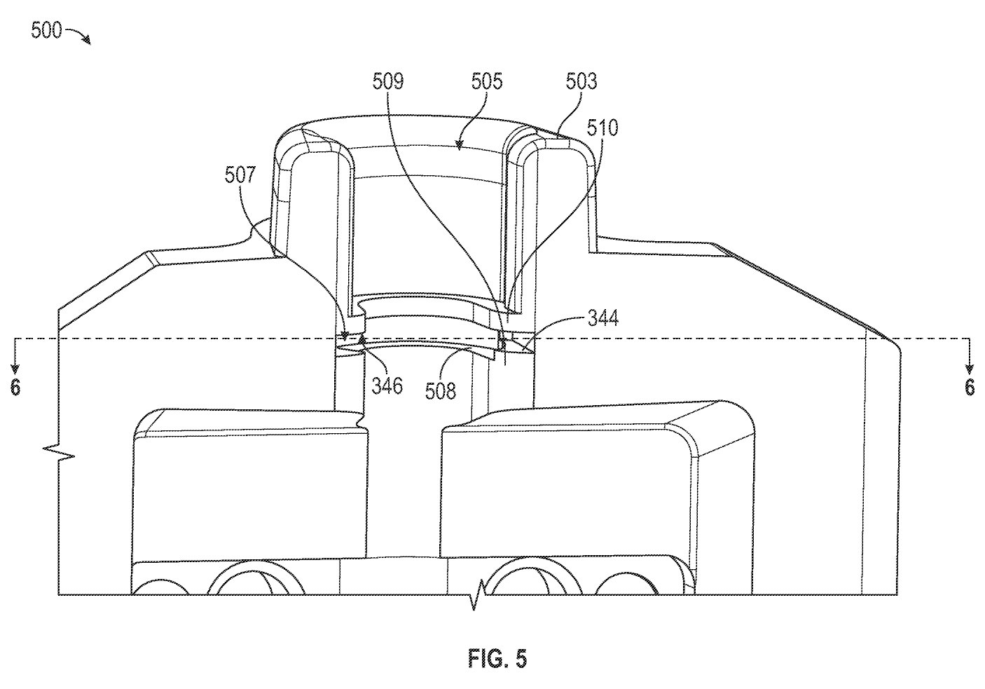

As described in detail below in reference to FIGS. 7-9, the body 305 is configured to be received into a mating recess of a fluid delivery device such that the bottom flange 321 is uniformly mated with a slot of the mating recess. In some implementations, the mating recess also includes an inner bore 508 and the bottom flange 321 is seated in the inner bore of the mating recess 505 (FIG. 5) only when the bottom flange 321 of the IV tubing fitment 310 is received within a slot 507 of the mating recess 505 preventing the IV tubing fitment 310 from being misloaded.

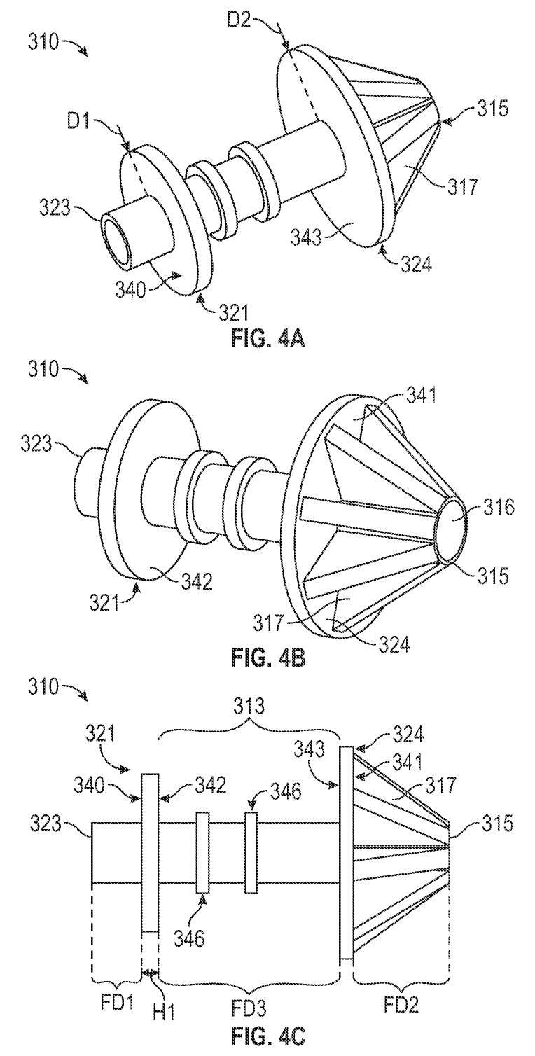

FIGS. 4A-4C illustrate different views of the IV tubing fitment 310 in accordance with some implementations. A first view (FIG. 4A) provides a bottom perspective view of the IV tubing fitment 310. The first view shows the body 305 including a first end 323, a bottom flange 321 adjacent to the first end 323, and a second end 315 adjacent to the bottom flange 321. The body 305 also includes a first opening 316 at the first end 323, a second opening 318 at the second end, and an inner surface forming a fluid pathway between the first opening 316 and the second opening 318.

The first end 323 is a cylindrically-shaped and configured to be fitted inside a tube 66 (FIG. 2). In some implementations, once the first end 323 has been fitted inside the tube 66, the tube 66 and the first end 323 are coupled such that they cannot be separated accidentally unless excessive force is applied. Excessive force, for purposes of this disclosure means, a force greater than accidental tugs and pulls and forces greater than those applied by the fluid delivery device. In some implementations, the first end 323 is molded within a portion of the tube 66 (when the body 305 is molded). In some implementations, the body 305 is configured to receive an end of a tube 66 in the first end 323 so that a portion of the tube 66 extends into the fluid pathway between the first opening 316 and the second opening 318.

A second view (FIG. 4B) provides a top perspective view of the IV tubing fitment 310. The second view shows the body 305 including the first end 323, the bottom flange 321, the second end 315, the top flange 324, and the plurality of wings 317. The body 305 includes the second opening 318 configured to receive an upstream fluid line 30 (FIG. 2). In some implementations, the body 305 receives the upstream fluid line 30 into the fluid pathway between the second opening 318 and the first opening 316. The upstream fluid line 30 is coupled with a sufficient amount of force to prevent the body 305 from being separated from the upstream fluid line 30 accidentally unless excessive force is applied. In some implementations, the body 305 is molded over a portion of the upstream fluid line 30 (when the body 305 is molded). By molding the body 305 over a portion of the upstream fluid line 30, the portion of the upstream fluid line 30 is prevented from accidentally being pulled out unless excessive force is applied.

As described above, the body 305 includes a plurality of wings 317 that extend from the outer surface of the body between the top flange 324 and the second end 315 to an upper surface of the top flange 324. The plurality of wings 317 terminate at the top flange 324 before the outer rim 319 along the perimeter of the top flange 324.

A third view (FIG. 4C) provides a side elevation view of the IV tubing fitment 310. The third view shows the body 305 including the first end 323, the bottom flange 321 adjacent to the first end 323, the second end 315, the top flange 324 adjacent to the second end 315 and spaced apart from the bottom flange 321. The top flange 324 is spaced apart from the bottom flange 321 with a middle portion 313 of the body extending therebetween.

FIGS. 4A-4C also illustrate the position of the bottom flange 321 and the top flange 324, relative to the first end 323 and the second end 315 of the body. Referring to the bottom flange 321, a lower surface 340 of the bottom flange faces toward and is spaced apart from the first end 323 by a first flange distance FD1 that is greater than zero. Thus, the bottom flange 321 is spaced apart from the first end 323 of the body. However, it should be understood that, in some embodiments of the present disclosure, the bottom flange 321 is aligned with the first end 323 such that the lower surface 340 of the bottom flange and the first end 323 are on a common plane therethrough.

Referring to the top flange 324, an upper surface 341 of the top flange faces toward and is spaced apart from the second end 315 by a second flange distance FD2 that is greater than zero. Thus, the top flange 324 is spaced apart from the second end 315 of the body. However, it should be understood that, in some embodiments of the present disclosure, the top flange 324 is aligned with the second end 315 such that the upper surface 341 of the top flange and the second end 315 are on a common plane therethrough.

The outer perimeter of each of the top flange 324 and the bottom flange 321 form a perimeter having a diameter, with the bottom flange 321 having a first diameter D1 and the top flange 324 having a second diameter D2. The first diameter D1 is configured to be received into a slot of the mating recess 505 of the fluid delivery device and is less than the second diameter D2. However, in some implementations of the present disclosure, the first diameter D1 is greater than the second diameter D2, or the first diameter D1 is equal to the second diameter D2.

The middle portion 313 of the body extends between the top flange 324 and the bottom flange 321 such that an upper surface 342 of the bottom flange and a lower surface 340 of the top flange are spaced apart by a third flange distance FD3 that is greater than zero. In some implementations of the present disclosure, the body includes one or more ribs 346 along the middle portion 313. The one or more ribs 346 are positioned between the bottom flange 321 and the top flange 324 and extend radially outward.

FIG. 5 illustrates a housing of a fluid delivery device, in accordance with some implementations. Fluid delivery device 500 is an instance of any infusion pump, described above in reference to FIGS. 1 and 2, configured to receive the IV tubing fitment 310. The fluid delivery device 500 includes a bezel housing 503 forming a mating recess 505 with a slot 507 and an inner bore 508 that is aligned with the slot 507. The slot 507 is formed by an inner surface of the fluid delivery device 500 such that, when the IV tubing fitment 310 is positioned in the mating recess 505, the bottom flange 321 is positioned in the slot 507.

The slot 507 is formed by portions of the fluid delivery device 500 forming a lower ledge 509 and an upper ledge 510 of the mating recess. The lower ledge 509 extends radially inward into the mating recess, and the upper ledge 510 extends radially inward into the mating recess. The lower ledge 509 comprises a top surface 344 facing toward the upper ledge 510, and the upper ledge 510 comprises a bottom surface 346 facing toward the lower ledge 509. To receive the bottom flange 321 of the IV tubing fitment laterally into the slot 507, the lower ledge 509 and the upper ledge 510 are spaced apart.

The lower ledge 509 and the upper ledge 510 are spaced apart to form a slot height S1 (FIG. 8) between top surface 344 of the lower ledge and the bottom surface 346 of the upper ledge. The slot height S1 is approximately equal to or greater than a height H1 of the bottom flange 321, where the height H1 of the bottom flange 321 is measured between the lower surface 340 and the upper surface 342 of the bottom flange.

After the IV tubing fitment 310 is inserted into the mating recess 505 and the bottom flange 321 is positioned within the slot 507, the IV tubing fitment 310 can be moved in a downward direction toward the lower ledge 509. As the IV tubing fitment 310 is moved downward, the bottom flange 321 can enter into the inner bore 508.

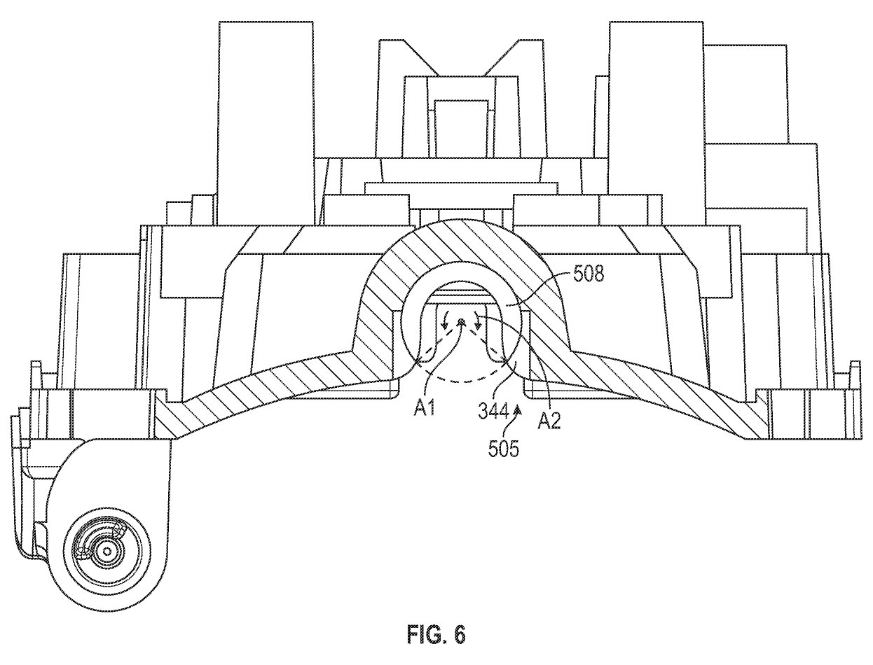

FIG. 6 illustrates a cross-sectional view of the fluid delivery device 500 of FIG. 5, taken along the line 6–6 of FIG. 5, showing the top surface 344 of the lower ledge and the inner bore 508. As can be seen, the inner bore 508 forms a sidewall with a perimeter that extends around a longitudinal axis of the mating recess A1. The perimeter of the inner bore extends around the longitudinal axis by an angle A2 that is greater than 180 degrees to resist lateral movement of the bottom flange 321. The perimeter the inner bore 508 also forms an inner bore diameter D3 that is approximately equal to or greater than the diameter D1 of the bottom flange.

FIG. 7 illustrates a first operational view of the IV tubing fitment 310 positioned adjacent to the mating recess 505 of the fluid delivery device. In particular, the door of the fluid delivery device 500 is open so that the mating recess 505 of the fluid delivery device can receive the IV tubing fitment 310. As shown in the first operation view, the IV tubing fitment 310 is configured so that the fluid delivery device 500 can only receive the IV tubing fitment 310 in a single configuration. More specifically, the bottom flange 321 can enter into the slot 507 due to the diameter D1 of the bottom flange being approximately equal to or less than the width W1 of the slot, and the slot height S1 being approximately equal to or greater than the height H1 of the bottom flange 321 (FIG. 8). Additionally, if a user attempted to insert the IV tubing fitment 310 in improper configuration, such as an inverted configuration, the plurality of wings 317 could engage against the housing and resist insertion of the IV tubing fitment 310 into the mating recess.

To insert or load the IV tubing fitment 310 into the fluid delivery device, the IV tubing fitment 310 is axially aligned with the mating recess 505 so that the bottom flange 321 is axially aligned with the slot 507, and top flange 324 is aligned with the bezel housing 503. In some implementations of the present disclosure, when the IV tubing fitment 310 is axially aligned with the mating recess 505, the top surface 344 of the lower ledge is aligned with the bottom surface 343 of the lower flange, and the bottom surface 346 of the upper ledge is aligned with the upper surface 342 of the lower flange. With the bottom flange 321 axially aligned with the slot 507, the IV tubing fitment 310 can be laterally moved into the mating recess such that the bottom flange 3210 is received into the slot 507.

Next, the IV tubing fitment 310 can be moved in a downward direction toward the lower ledge 509. As the IV tubing fitment 310 is moved downward, the bottom flange 321 at least partially enters into the inner bore 508.

FIG. 8 illustrates a second operational view of the IV tubing fitment 310 loaded in the mating recess 505 with the bottom flange 321 positioned within the inner bore 508. FIG. 9 illustrates a cross-sectional view of the IV tubing fitment 310 and the fluid delivery device 500, taken along the line 9–9 of FIG. 8, with the bottom flange 321 positioned within the inner bore 508. With the IV tubing fitment 310 positioned with the bottom flange 321 positioned within the inner bore 508, lateral movement of the IV tubing fitment 310 is resisted. Stated in another manner, when the IV tubing fitment 310 is moved laterally, relative to the fluid delivery device, the outer perimeter of the bottom flange 321 can engage against the perimeter of the inner bore 508 formed by the lower ledge, thereby resisting lateral movement of the IV tubing fitment 310 in the mating recess.

When the IV tubing fitment 310 is loaded into the mating recess of the fluid delivery device, misloading or misalignment of the IV tubing fitment relative to the fluid delivery device is resisted or prevented. More specifically, undesirable tilting of IV tubing fitment 310 and axial movement of the IV tubing fitment 310 in the mating recess is resisted, thereby preventing damage to the IV tubing fitment when a door of the fluid delivery device is closed and the improving the accuracy and consistency of the flow rate generated by the fluid delivery device.

To remove the IV tubing fitment 310, the door of the fluid delivery device 500 is opened, and the IV tubing fitment 310 can be raised in an upward direction, toward the upper ledge 510 so that the bottom flange 321 is axially aligned with the slot 507. Next, the IV tubing fitment 310 can be laterally moved in a direction away from the mating recess.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

Illustration of Subject Technology as Clauses: Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An intravenous (IV) tubing fitment, comprising: a body comprising: a first end configured to couple to a first portion of a tubing; a second end configured to receive a second portion of the tubing; a bottom flange adjacent to the first end; and a top flange adjacent to the second end; wherein the body is configured to be received into a mating recess of a fluid delivery device such that the bottom flange is positioned within a slot in the mating recess and engagement of the body against the fluid delivery device resists tilting of the body relative to the fluid delivery device.

Clause 2. wherein the body is unitary.

Clause 3. The IV tubing fitment of any of Clause 1 or Clause 2, wherein the body comprises a plurality of wings extending from the top flange toward the second end.

Clause 4. The IV tubing fitment of any of Clause 1 through Clause 3, wherein engagement of the body against the fluid delivery device resists axial and lateral movement of the body relative to the fluid delivery device.

Clause 5. The IV tubing fitment of any of Clause 1 through Clause 4, wherein the bottom flange comprises a upper surface and a lower surface, the upper surface facing toward the second end of the body, and the lower surface facing toward the first end of the body.

Clause 6. The IV tubing fitment of Clause 5, wherein the bottom flange is spaced apart from the first end of the body forming a flange distance between the lower surface of the bottom flange and the first end of the body, and wherein the flange distance is greater than zero.

Clause 7. The IV tubing fitment of Clause 5, wherein the bottom flange comprises an outermost perimeter forming a bottom flange diameter, and the body comprises a middle portion, between the bottom flange and the top flange, forming a middle portion diameter, and wherein the bottom flange diameter is greater than the middle portion diameter.

Clause 8. The IV tubing fitment of any of Clause 1 through Clause 7, wherein the top flange is spaced apart from the bottom flange by a middle portion of the body.

Clause 9. The IV tubing fitment of any of Clause 1 through Clause 8, wherein the body comprises an inner surface forming a fluid passage extending through the first end and the second end.

Clause 10. An intravenous (IV) tubing fitment system comprising: an IV tubing fitment comprising a body having a first end, configured to couple to a first portion of a tubing, a second end, configured to receive a second portion of the tubing, a bottom flange, and a top flange, wherein the bottom flange is adjacent to the first end, and the top flange adjacent to the second end; and a fluid delivery device comprising a housing having an inner surface forming a mating recess comprising a lower ledge, extending radially inward into the mating recess, an upper ledge, extending radially inward into the mating recess, and a slot formed between the lower ledge and the upper ledge; wherein, the body is configured to be received into the mating recess such that the bottom flange is positioned within the slot and a middle portion of the body, between the bottom flange and the top flange extends through a bore of the upper ledge, and wherein engagement of the body against the fluid delivery device resists tilting of the body relative to the fluid delivery device.

Clause 11. The IV tubing fitment system of Clause 10, wherein the lower ledge comprises a top surface facing toward the upper ledge, and an inner bore extends into the lower ledge, in a direction away from the upper ledge.

Clause 12. The IV tubing fitment system of Clause 11, wherein, when the bottom flange is received into the slot, engagement of an outermost perimeter of the bottom flange against the lower ledge resists lateral movement of the body relative to the housing.

Clause 13. The IV tubing fitment system of Clause 11, wherein the inner bore comprises an inner bore diameter, and the bottom flange comprises an outermost perimeter forming a bottom flange diameter, and wherein the inner bore diameter is approximately equal to or greater than the bottom flange diameter.

Clause 14. The IV tubing fitment system of Clause 13, wherein the inner bore comprises a perimeter that extends over a recess angle that is greater than 180 degrees.

Clause 15. The IV tubing fitment system of any of Clause 13 through Clause 14, wherein the lower ledge comprises a top surface facing toward the upper ledge, the upper ledge comprises a bottom surface facing toward the lower ledge, and a slot height is formed between the top surface of the lower ledge and the bottom surface of the upper ledge, and wherein the slot height is approximately equal to or greater than a height of the bottom flange.

Clause 16. The IV tubing fitment system of any of Clause 13 through Clause 15, wherein, when the bottom flange is received into the slot, engagement of the bottom flange against any of the upper ledge or lower ledge resist axial movement and tilting movement of the body relative to the housing.

Clause 17. The IV tubing fitment system of any of Clause 13 through Clause 16, wherein, when the body is received into the mating recess, the body is configured to rotate about a longitudinal axis of the body in a clockwise direction and a counterclockwise direction, relative to the fluid delivery device.

Clause 18. A method of providing an intravenous (IV) tubing fitment system, the method comprising: providing an IV tubing fitment comprising a body having a first end, configured to couple to a first portion of a tubing, a second end, configured to receive a second portion of the tubing, a bottom flange, and a top flange, wherein the bottom flange is adjacent to the first end, and the top flange adjacent to the second end; providing a fluid delivery device comprising a housing having an inner surface forming a mating recess comprising a lower ledge, extending radially inward into the mating recess, an upper ledge, extending radially inward into the mating recess, and a slot formed between the lower ledge and the upper ledge; and positioning the body into the mating recess such that the bottom flange is within the slot and a middle portion of the body, between the bottom flange and the top flange extends through a bore of the upper ledge, and engagement of the body against the fluid delivery device resists tilting of the body relative to the fluid delivery device.

Clause 19. The method of Clause 18, further comprising inserting the bottom flange into an inner bore of the lower ledge such that such that engagement of an outermost perimeter of the bottom flange against the lower ledge resists lateral movement of the body relative to the housing.

Clause 20. The method of any of Clause 18 or Clause 19, wherein further comprising closing a door, forming another part of the mating recess, after positioning the body into the mating recess.

Further Consideration: In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such an implementation may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled,” or the like may refer to being directly coupled. In another aspect, the term “coupled,” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various implementations for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. An intravenous (IV) tubing fitment, comprising:
a body comprising:
a first end configured to couple to a first portion of a tubing;
a second end configured to receive a second portion of the tubing;
a bottom flange adjacent to the first end; and
a top flange adjacent to the second end;
wherein the body is configured to be received into a mating recess of a fluid delivery device such that the bottom flange is positioned within a slot in the mating recess and engagement of the body against the fluid delivery device resists tilting of the body relative to the fluid delivery device.

2. The IV tubing fitment of claim 1, wherein the body is unitary.

3. The IV tubing fitment of claim 1, wherein the body comprises a plurality of wings extending from the top flange toward the second end.

4. The IV tubing fitment of claim 1, wherein engagement of the body against the fluid delivery device resists axial and lateral movement of the body relative to the fluid delivery device.

5. The IV tubing fitment of claim 1, wherein the bottom flange comprises a upper surface and a lower surface, the upper surface facing toward the second end of the body, and the lower surface facing toward the first end of the body.

6. The IV tubing fitment of claim 5, wherein the bottom flange is spaced apart from the first end of the body forming a flange distance between the lower surface of the bottom flange and the first end of the body, and wherein the flange distance is greater than zero.

7. The IV tubing fitment of claim 5, wherein the bottom flange comprises an outermost perimeter forming a bottom flange diameter, and the body comprises a middle portion, between the bottom flange and the top flange, forming a middle portion diameter, and wherein the bottom flange diameter is greater than the middle portion diameter.

8. The IV tubing fitment of claim 1, wherein the top flange is spaced apart from the bottom flange by a middle portion of the body.

9. The IV tubing fitment of claim 1, wherein the body comprises an inner surface forming a fluid passage extending through the first end and the second end.

10. An intravenous (IV) tubing fitment system comprising:
an IV tubing fitment comprising a body having a first end, configured to couple to a first portion of a tubing, a second end, configured to receive a second portion of the tubing, a bottom flange, and a top flange, wherein the bottom flange is adjacent to the first end, and the top flange adjacent to the second end; and
a fluid delivery device comprising a housing having an inner surface forming a mating recess comprising a lower ledge, extending radially inward into the mating recess, an upper ledge, extending radially inward into the mating recess, and a slot formed between the lower ledge and the upper ledge;
wherein, the body is configured to be received into the mating recess such that the bottom flange is positioned within the slot and a middle portion of the body, between the bottom flange and the top flange extends through a bore of the upper ledge, and wherein engagement of the body against the fluid delivery device resists tilting of the body relative to the fluid delivery device.

11. The IV tubing fitment system of claim 10, wherein the lower ledge comprises a top surface facing toward the upper ledge, and an inner bore extends into the lower ledge, in a direction away from the upper ledge.

12. The IV tubing fitment system of claim 11, wherein, when the bottom flange is received into the slot, engagement of an outermost perimeter of the bottom flange against the lower ledge resists lateral movement of the body relative to the housing.

13. The IV tubing fitment system of claim 11, wherein the inner bore comprises an inner bore diameter, and the bottom flange comprises an outermost perimeter forming a bottom flange diameter, and wherein the inner bore diameter is approximately equal to or greater than the bottom flange diameter.

14. The IV tubing fitment system of claim 13, wherein the inner bore comprises a perimeter that extends over a recess angle that is greater than 180 degrees.

15. The IV tubing fitment system of claim 10, wherein the lower ledge comprises a top surface facing toward the upper ledge, the upper ledge comprises a bottom surface facing toward the lower ledge, and a slot height is formed between the top surface of the lower ledge and the bottom surface of the upper ledge, and wherein the slot height is approximately equal to or greater than a height of the bottom flange.

16. The IV tubing fitment system of claim 10, wherein, when the bottom flange is received into the slot, engagement of the bottom flange against any of the upper ledge or lower ledge resist axial movement and tilting movement of the body relative to the housing.

17. The IV tubing fitment system of claim 10, wherein, when the body is received into the mating recess, the body is configured to rotate about a longitudinal axis of the body in a clockwise direction and a counterclockwise direction, relative to the fluid delivery device.

18. A method of providing an intravenous (IV) tubing fitment system, the method comprising:
providing an IV tubing fitment comprising a body having a first end, configured to couple to a first portion of a tubing, a second end, configured to receive a second portion of the tubing, a bottom flange, and a top flange, wherein the bottom flange is adjacent to the first end, and the top flange adjacent to the second end;
providing a fluid delivery device comprising a housing having an inner surface forming a mating recess comprising a lower ledge, extending radially inward into the mating recess, an upper ledge, extending radially inward into the mating recess, and a slot formed between the lower ledge and the upper ledge; and
positioning the body into the mating recess such that the bottom flange is within the slot and a middle portion of the body, between the bottom flange and the top flange extends through a bore of the upper ledge, and engagement of the body against the fluid delivery device resists tilting of the body relative to the fluid delivery device.

19. The method of claim 18, further comprising inserting the bottom flange into an inner bore of the lower ledge such that such that engagement of an outermost perimeter of the bottom flange against the lower ledge resists lateral movement of the body relative to the housing.

20. The method of claim 18, further comprising closing a door, forming another part of the mating recess, after positioning the body into the mating recess.
